SEQUENCE LISTING (16 sequences)

SEQ ID NO: 1 - RNA, 336 nt, Pneumocystis carinii
gagggucaugaaagcggcgugaaaacguuagcuagugaucuggaauaaauucagauugcgacacugucaaauugcggggaagcccuaaagauucaacuacuaagcaguuuguggaaacacagcuguggccgaguuaauagcccuggguauaguaacaauguugaauaugaaucuuuugcgagaugaaaugggugauccgcagccaaguccuaagggcauuuuugucuauggaugcaguucaacgacuagauggcaguggguauuguaaggaauugcaguuuucuugcagugcuuaagguauagucuauccucuuucgaaagaaagaguauaucuag

SEQ ID NO: 2 - RNA, 18 nt, Artificial Sequence
  Description of Artificial Sequence Synthetic oligonucleotide
augacucucgugaaacau

SEQ ID NO: 3 - RNA, 12 nt, Artificial Sequence
  Description of Artificial Sequence Synthetic oligonucleotide
augacuaaacau

SEQ ID NO: 4 - RNA, 12 nt, Artificial Sequence
  Description of Artificial Sequence Synthetic oligonucleotide
cucgugaaacau

SEQ ID NO: 5 - RNA, 30 nt, Pneumocystis carinii
auccucuuucgaaagaaagaguauauugug

SEQ ID NO: 6 - RNA, 30 nt, Pneumocystis carinii
auccucuuucgaaagaaagaguauaucuag

SEQ ID NO: 7 - RNA, 13 nt, Artificial Sequence
  Description of Artificial Sequence Synthetic oligonucelotide
gcucgugaaacau

SEQ ID NO: 8 - RNA, 10 nt, Artificial Sequence
  Description of Artificial Sequence Synthetic oligonucelotide
cuagcucgug

SEQ ID NO: 9 - RNA, 10 nt, Artificial Sequence
  Description of Artificial Sequence Synthetic oligonucelotide
cuaacucgug

SEQ ID NO: 10 - RNA, 16 nt, Artificial Sequence
  Description of Artificial Sequence Synthetic oligonucelotide
cuagcucgugaaacau

SEQ ID NO: 11 - DNA, 25 bp, Artificial Sequence
  Description of Artificial Sequence Synthetic primer
ctctaatacgactcactatagaggg

SEQ ID NO: 12 - DNA, 28 bp, Artificial Sequence
  Description of Artificial Sequence Synthetic primer
cacaatatactctttctttcgaaagagg

SEQ ID NO: 13 - DNA, 28 bp, Artificial Sequence
  Description of Artificial Sequence Synthetic primer
ttagatatactctttctttcgaaagagg

SEQ ID NO: 14 - DNA, 34 bp, Artificial Sequence
  Description of Artificial Sequence Synthetic primer
cacgagctagatatactctttctttcgaaagagg

SEQ ID NO: 15 - DNA, 34 bp, Artificial Sequence
  Description of Artificial Sequence Synthetic primer
cacgagttagatatactctttctttcgaaagagg

SEQ ID NO: 16 - DNA, 40 bp, Artificial Sequence
  Description of Artificial Sequence Synthetic primer
atgtttcacgagctagatatactctttctttcgaaagagg

(19) United States
(12) Patent Application Publication
(10) Pub. No.: US 2007/0270367 A1
(43) Pub. Date: Nov. 22, 2007

(54) Rybozyme-catalyzed insertion of targeted sequences into RNA

(75) Inventors: Stephen M. Testa, Lexington, KY (US); Ashley K. Johnson, Richmond, VA (US)

Correspondence Address: BUCHANAN, INGERSOLL & ROONEY PC, POST OFFICE BOX 1404, ALEXANDRIA, VA, 22313-1404, US

(73) Assignee: University of Kentucky Research Foundation, Lexington, KY (US)

(21) Appl. No.: 11/653,303

(22) Filed: Jan. 16, 2007

Related U.S. Application Data
(63) Continuation of application No. 11/435,825, filed on May 18, 2006.
(60) Provisional application No. 60/682,021, filed on May 18, 2005.

(51) Int. Cl. A61K 31/7105; C07H 21/02; C12Q 1/68

(52) U.S. Cl. 514/44; 435/6; 536/23.1

(57) ABSTRACT

Group I intron-derived ribozymes can be modified to perform a reaction called trans insertion-splicing (TIS) where the ribozyme binds two exogenous RNA substrates and inserts a sequence from one directly into the other. Reaction products are stable, with no visible loss at extended times. The ribozyme recognizes the two substrates primarily through base pairing and utilizes an ωG on the ribozyme and a 3′-G on the sequence being inserted. The internal guide sequence of the ribozyme is utilized to sequentially bind both substrates, forming independent P1 helices. The reaction can also be performed without a first substrate, where the ribozyme is made with the insert sequence appended to its 3′ end so as to perform a single substrate insertion targeted to any RNA sequence.

### CROSS REFERENCE TO RELATED APPLICATION

This application is a continuation application of U.S. Ser. No. 11/435,825, filed May 18, 2006 which claims priority under 35 U.S.C. §119 to U.S. Provisional Application No. 60/682,021 filed May 18, 2005, the entire content of which is hereby incorporated herein by reference.

### BACKGROUND

1. Field of the Invention

The invention relates to methods and reagents for the manipulation of RNA sequences.

2. Description of the Related Art

Since the discovery of catalytic RNA in the early 1980s (1, 2), the view of RNA as simply an intermediary between DNA and proteins has drastically changed. For example, many naturally occurring catalytic RNA reactions have been found (3, 4). Ribozymes derived from naturally occurring catalytic RNAs have been developed for use as therapeutics (5-9) and biochemical tools (10, 11). Even more recently, the discovery of RNA interference (12, 13) and riboswitches (14, 15) show that RNA is involved in the most basic levels of gene regulation.

### SUMMARY OF THE INVENTION

A TIS ribozyme for inserting a sequence from a first insert substrate into a target location in a second target substrate can comprise an RNA sequence derived from an isolated group I intron such that the ribozyme has a modified non-native internal guide sequence (IGS) and a 3′ ωG. The ribozyme can be derived from a group I intron of P. carinii, for example the ribozyme can be derived from the sequence of P. carinii group I intron rP-8/4. In some cases, an insert sequence having a 3′ ωGi can be appended to the 3′ ωG of the ribozyme, in which case a first insert substrate is not needed for insertion as the ribozyme will be capable of inserting the insert sequence appended at its 3′ end into the sequence targeted by the non-native IGS.

A mixture for performing the reaction will comprise a TIS ribozyme as described above and an RNA target substrate having a sequence that is complementary to a segment of the non-native IGS sequence of the ribozyme. Where the ribozyme is not provided with as insert sequence attached at its 3′ end, the reaction mixture can include an insert substrate that comprises an insert sequence that is complementary to a segment of the non-native IGS sequence of the ribozyme and has a 3′ G.

A method of inserting an RNA insert sequence into an RNA target substrate can be performed by contacting the target substrate with a TIS ribozyme, where the non-native IGS sequence of the ribozyme contains a segment that is complimentary to residues of a target sequence of the target substrate on both sides of an insertion site except for a mismatched base pairing at the insertion site. The method can also include contacting the ribozyme with an insert substrate wherein the non-native IGS sequence of the ribozyme comprises a segment that is complimentary to a segment of an insert sequence of the insert substrate.

Where it is desirable to stop translation of an mRNA, for example to prevent expression or to stop runaway expression, the insert sequence can comprise a stop codon. In this case, the non-native IGS sequence of a TIS ribozyme can comprise a segment complimentary to a stop codon for reactions, or a stop codon can be contained on an insert sequence that is provided already attached to the 3′ end of the ribozyme.

A method of inserting an RNA insert sequence into an RNA target substrate can be performed in vivo by introducing a nucleic acid comprising an expression cassette which includes a sequence encoding the ribozyme into a cell. The nucleic acid is preferably a DNA molecule containing an expression cassette with a promoter operably-linked to an isolated nucleotide sequence encoding a TIS ribozyme. Such a method can be used as a research tool or therapeutically as a method of treating a disease associated with a mutation of a gene that results in production of a non-native mRNA that is missing a segment normally found in a native mRNA produced from the gene. In a therapeutic method, a nucleic acid comprising an expression cassette which includes a sequence encoding a TIS ribozyme can be introduced into cells of a patient, where the TIS ribozyme is designed such that the IGS sequence contains a sequence complementary to residues on both sides of the site of the missing segment of the non-native mRNA and a base pair mismatch at the site of the missing segment of the non-native mRNA can be administered to a patient possessing the mutation. The ribozyme comprises an insert sequence appended to the 3′ ωG that can restore substantial functionality to the mRNA. Alternatively, in a therapeutic method, a TIS ribozyme can be administered directly to a patient possessing the mutation, for example via injection, inhalation, or the like. The insert sequence can include the native sequence of the segment missing from the non-native mRNA or a segment or its genetic code equivalent, an insert sequence can restore the reading frame of the mRNA, or the insert sequence can replace a missing or destroyed stop codon.

### BRIEF DESCRIPTION OF THE DRAWINGS

FIG. 1. The trans excision-splicing reaction and trans insertion-splicing reactions are compared. A) The 36mer TES starting material reacts with ribozyme rPC to give the 16mer TES product and 20mer excised region (6). B) The 12mer starting material and 9mer insert react with ribozyme rPC to give the 18mer TIS product.

FIG. 2. Proposed secondary structure of a P. carinii group I intron-derived ribozyme (rPC) (SEQ ID NO: 1).

FIG. 3. Results demonstrating the first nucleophilic attack in the TIS reaction. Polyacrylamide gel showing reactants and products of the TIS reaction using 200 nM ribozyme, 1 μM insert, 1 nM starting material, and 10 mM MgCl₂ run for 2 h at 44° C. Lanes E and I contain 5′-end radiolabeled 6mer, 9mer, 12mer, and 18mer (sequences in Table 1) run as a size control. Lane L contains 3′-end radiolabeled 9mer run as a size control. Lanes A and B contain the standard TIS reaction run with rPC ribozyme, 9mer insert, and 5′-end radiolabeled 12mer (lane A) or 3′-end radiolabeled 12mer (lane B). The starting material was radiolabeled on either the 5′ or 3′-end for reactions run in lanes A, B, C, D, F, G, and H. The insert was radiolabeled on either the 5′ or 3′-end for reactions run in lanes J, K, M, N, and O and are not at optimum concentrations for TIS. The radiolabeled end is indicated at the top of the gel.

FIG. 4. Sequencing of 5′-end radiolabeled TIS product. The 18mer TIS product (SEQ ID NO: 2) was isolated from a large reaction of rPC, 9mer insert, and 5′-end radiolabeled 12mer starting material. Both synthetic 18mer product and isolated 18mer TIS product were enzymatically sequenced next to each other using U2, CL-3, T1, and B. cer. endonucleases. The dotted line shows the position of the inserted region (CUCGUG) between the 5′ and 3′-ends of the 12mer starting material. Nuclease U2 is specific for adenosine, CL-3 for primarily cytidine, T1 for guanosine, and B. cer. for primarily cytidine and uridine. Note that the intensity of the bands in the boxed regions was enhanced relative to the rest of the gel for easier visualization.

FIG. 5. TIS reaction characterization graphs. Except for the changing variable, TIS reactions were run under reaction conditions of 200 μM rPC ribozyme, 1 μM 9mer insert, and 10 mM MgCl₂ using 1 nM 5′-end radiolabeled 12mer starting material for 2 h at 44° C. Each graph represents the average of two independent assays. The standard deviations for each point in the graphs were under twenty percent.

FIG. 6. A representative time-course study gel and observed rate constants for the TIS reaction. The polyacrylamide gel shows reactants and products of the TIS reaction using 200 nM ribozyme (rPC), 1 μM insert (9mer), 1 nM 5′-end radiolabeled starting material (12mer), and 10 mM MgCl₂ run for 3 h at 44° C. The graphs plot the average amount of 18mer TIS product from two independent time studies run in 6, 10, 14, or 18 mM MgCl₂. The standard deviations for each point in the graphs were under twenty percent.

FIG. 7. Proposed mechanism for the trans insertion-splicing reaction. The rPC ribozyme is represented by black lines, and the IGS sequence and ωG (in bold) are shown. The insert sequence is shown in white letters with a gray background. The ωGi in the insert is distinguished with a black background. A) The rPC ribozyme binds the 9mer insert (pathway “a”). Alternatively, the 12mer (SEQ ID NO: 3) starting material (pathway “b”) can bind the ribozyme to give the dead-end products. B) The insert (highlighted in gray and black) forms P1i and P10 helices with the IGS of the ribozyme. The ωG in the ribozyme (in bold) attacks at the 5′-splice site in the 9mer insert. C) The 12mer starting material (SEQ ID NO: 3) displaces the insert fragments from P1i and forms a second P1 helix. A nucleophilic attack by the ωGi on the insert (attached to the ribozyme, shown with black background) occurs at the 5′-splice site in the 12mer starting material. D) A nucleophilic attack of the 3′-U (SEQ ID NO: 4) from the 5′-half of the starting material at the ωG in the ribozyme (in bold) produces the 18mer TIS product (SEQ ID NO: 2) (inserted region with gray and black background in between the 5′ and 3′ halves of the 12mer starting material). E) This alternative pathway involves the 12mer starting material (SEQ ID NO: 3) forming the P1 helix first. The ωG on the ribozyme (in bold) can attack at the 5′-splice site in the 12mer, leading to 6mer product and the 3′-half of the 12mer attached to the ribozyme.

FIG. 8. Proof for the second and third nucleophilic attacks in the TIS reaction. Polyacrylamide gel showing reactants and products of the TIS reaction using 200 nM ribozyme, 1 μM insert, 1 nM starting material, and 10 mM MgCl₂ run for 2 h at 44° C. Lanes E, I, and L contain 5′-end radiolabeled 6mer, 9mer, 12mer, and 13mer (sequences in Table 1) run as a size control. Note that reactions in lanes F, G, H, J, K, M, N, and O were run without added insert since the insert sequence was attached to the ribozyme intermediate. Lanes A and B contain the standard TIS reaction run with rPC ribozyme, 9mer insert, and 5′-end radiolabeled 12mer starting material (lane A) or 3′-end radiolabeled 12mer starting material (lane B). The rest of the lanes are described in the text. The starting material was radiolabeled on either the 5′ or 3′-end for all the reactions. Note that lanes M, N, and O were run with the intermediate 6mer starting material. The radiolabeled end is indicated at the top of the gel.

FIG. 9. Secondary structure at the 3′-end of the rPC-1 (SEQ ID NO: 5) and rPC (SEQ ID NO: 6) ribozymes. The rPC-1 ribozyme is the exact sequence of the P. carinii group I intron, capable of forming a P9.0 helix. The rPC ribozyme used in the TIS reaction differs from rPC-1 by three nucleotides and cannot form a P9.0 helix.

### DETAILED DESCRIPTION OF THE PREFERRED EMBODIMENTS OF THE INVENTION

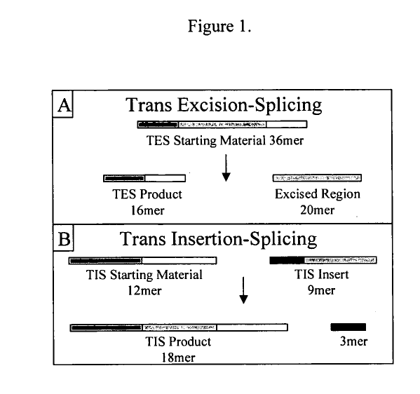

The inherent binding and catalytic activity of group I intron-derived ribozymes can be exploited to catalyze reactions that modify RNA. U.S. patent application Ser. No. 10/730,261, incorporated herein by reference in its entirety, describes a trans excision-splicing (TES) reaction, developed with a P. carinii group I intron-derived ribozyme, which can bind an exogenous RNA substrate, remove a section from the middle, and splice the ends back together (FIG. 1A) (6, 16, 17). Described herein is a new group I intron-derived ribozyme reaction called trans-insertion-splicing (TIS), whereby in a two-substrate reaction, a first RNA substrate (comprising an insert sequence) can be inserted into a second RNA substrate (comprising a target sequence). The reaction can also be utilized in a one-substrate mode by providing a group I intron derived ribozyme that comprises an insert sequence to be inserted directly into a specific target RNA substrate.

The two-substrate TIS reaction provides the first example of the direct insertion of one exogenous RNA substrate into a second exogenous RNA substrate using a ribozyme that can be substantially unaltered from its intronic form, except for modifications to the internal guide sequence (IGS). In the two-substrate TIS reaction, the ribozyme apparently utilizes the IGS to separately bind both exogenous substrates, forming two independent P1 helices. In addition, the ribozyme appears to sequentially position the ωG of the ribozyme and a 3′-G on the insert (attached to the ribozyme and presumably acting as the ωG of the ribozyme) into the guanosine binding site (GBS) of the ribozyme.

A ribozyme for the reaction can be derived from an isolated group I intron ribozyme with minimal modification. As an example, for the two-substrate TIS reaction to splice an insert sequence from a first substrate into a target sequence in a second separate substrate, the ribozyme can comprise a ribozyme derived from a group I intron having an IGS at its 5′ end that is different from its native IGS and an ωG at its 3′ end. The IGS is modified to comprise a first segment that is complementary to the target RNA substrate at the site of insertion and a second segment, which can be the same as or overlapping with the first segment, that is complementary to the insert sequence. The non-native IGS preferably contains nucleic acid sequences complimentary to about 4, 5, 6, 7, 8, 9 or more bases of the target substrate and insert substrate including a base pairing mismatch that defines the splice site within the complementary sequences. The insert sequence substrate also comprises a 3′ G, designated ωGi. If desired, the ribozyme can be modified to prevent formation of a P9.0 structure.

Alternatively, a ribozyme capable of inserting a sequence into a specific location in an RNA target can be made with the insert sequence attached. In this case, the ribozyme can have an RNA sequence derived from a group I intron with the desired RNA insert sequence attached to the ωG at the 3′ end of the ribozyme and the 3′ end of the insert sequence comprises a ωGi. The ribozyme also comprises an internal guide sequence (IGS) at its 5′ end that is different from the native IGS that is complementary to about 4, 5, 6, 7, 8, 9 or more bases of the target substrate with a base-pairing mismatch at the site of insertion.

A simple schematic of the two-substrate TIS reaction is shown in FIG. 1B. The ability of a group I intron-derived ribozyme to perform the TIS reaction has been proven by enzymatic sequencing of insertion products. A three-step mechanism has been elucidated using modified substrates to trap isolated reaction steps. Although the same P. carinii ribozyme was used to develop the TIS reaction as was used to develop the TES reaction (FIG. 1A), the TIS reaction is not simply the reverse of the TES reaction.

Our discovery shows that group I intron-derived ribozymes are capable of more dynamic and complex molecular interactions than previously demonstrated. The proposed mechanism for the TIS reaction and various exemplary uses for group I intron-derived ribozyme reactions are discussed in greater detail below. A large number of group I intron ribozymes have been reported. This reaction was tested using a ribozyme, rP-8/4x, from the opportunistic pathogen Pneumocystis carini and can be performed using a form of this ribozyme in which the targeting sequence is modified. It will also be appreciated that while this reaction was developed and demonstrated using a group I intron-derived ribozyme from P. carinii, the reaction is not limited to this exemplary ribozyme. It is expected that the reaction should be capable of being performed using almost any isolated group I intron-derived ribozyme that has been modified as described below. Preferably, a group I intron-derived ribozyme for use in the methods described below comprises the features which are identified below as participating in the TIS mechanism.

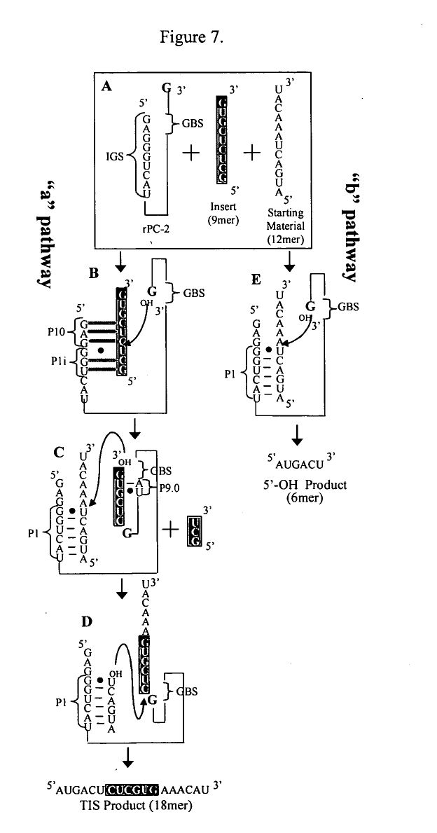

The TIS Mechanism. Evidence suggests there are three reaction steps in forming a TIS product as illustrated in FIG. 7. The requirements of the reaction are a group I derived ribozyme having an ωG at the 3′ end and an IGS sequence at the 5′ end that is complementary to a target sequence on a starting material and which provides for a wobble pair (i.e. a base pairing mismatch) that will define the splice site. An RNA starting material substrate comprises the target sequence for the insertion. An insert substrate is complementary to a segment of the IGS except for a wobble pair and also comprises a G at its 3′ end, which will be designated ωGi. The portions of the IGS that are complementary to the target sequence and insert sequence need not be the same, the sequences can also overlap. Preferably, the number of residues on the IGS that are complementary to the target and insert sequences is sufficient to provide specificity and comprises at least 4 or 5 base pairing residues plus the wobble pairs.

Step 1: The insert (such as the 9mer of the examples) binds the IGS of the ribozyme forming a P1i helix. The insert forms a wobble pair defining a splice site such as marked with a dot in FIG. 7, panel B and the ωG on the ribozyme then attacks at the newly formed 5′-splice site. The 5′ end of the insert substrate is released and the 3′ end of the insert substrate is now appended to the 3′ end of the ribozyme. In the example, this gives GCU+rPC-ωGCUCGUG+AUGACUAAACAU (SEQ ID NO: 3) (FIG. 7B). This reaction step appears to be the same as the reverse of the second step of group I intron self-splicing (20), and has been seen in other ribozyme reactions (21-24).

Step 2: The starting material (such as the 12mer of the example) displaces the insert fragment from the IGS and subsequently forms another P1 helix. The ωGi from the insert (now attached to the 3′-end of the ribozyme) attacks at a wobble pair at the newly formed second 5′-splice site. In the example, this gives AUGACU+rPC-ωGCUCGUGAAACAU (SEQ ID NO: 7) (FIG. 7C). This step is similar to the first reaction step (described above).

Step 3: The free 3′-OH at the splice site of the target sequence (the 3′-U of AUGACU in the example) can then attack at the ωG of the ribozyme intermediate (rPC-ωGCUCGUGAAACAU) (SEQ ID NO: 7), releasing the TIS product. In the example this gives the AUGACUCUCGUGAAACAU (SEQ ID NO: 2) 18mer. (FIG. 7D). This reaction step appears to be the same as the second step of group I intron self-splicing (20).

No experiments have contradicted this proposed mechanism. It may be noted that a competing reaction occurs when the 12mer starting material forms the P1 helix first (before the insert forms the P1i helix). This reaction results in the formation of truncated starting material. In the example, this gives the 6mer AUGACU. And the 3′-end of the starting material becomes attached to the 3′-end of the ribozyme (FIG. 7E). In the example, this leaves the AAACAU attached to the ribozyme, which has no ωG and therefore cannot complete the TIS reaction.

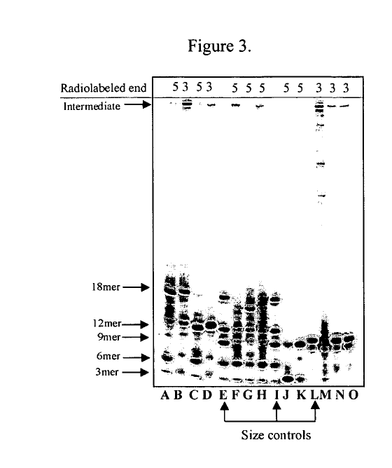

The TIS reaction as with other ribozyme reactions (21-24), is efficient only where the ribozyme ends in G (which is called ωG). It must be noted that there is evidence that when T7 RNA polymerase is used to synthesize RNA, it frequently adds one or two template-independent nucleotides onto the 3′-end of RNA transcripts (25). There is no simple way to ensure that extra nucleotides are not being adding to ribozymes that are made by in vitro transcription. When a ribozyme ending in ωA is synthesized, it substantially prevents reactivity (FIG. 3, lane C). It has been reported that the extra nucleotides added by T7 RNA polymerase tend to be mostly As and Cs (25, 26). However, although template-independent nucleotides may be added to the TIS ribozymes when made by T7 RNA polymerase, and the added nucleotides are not predominantly Gs, it appears that they are not prevalent.

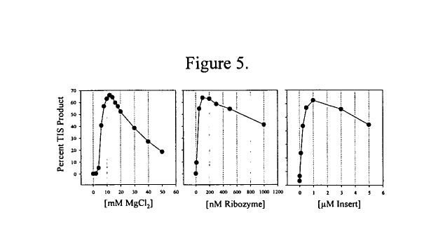

Factors that Influence the TIS Reaction. The TIS reaction was characterized for both yield and rate under various conditions. Preferable reaction conditions using the exemplary substrates were found to be about 10 mM MgCl₂, 200 nM rPC ribozyme, and 1 μM 9mer insert, using 1 nM radiolabeled starting material (FIG. 5). This represents a 200:1000:1 ratio of ribozyme, insert, and starting material. The relative proportions of these reaction components can be fairly specific, such that decreasing or increasing the concentrations of insert or ribozyme gives decreased TIS product yields. At ribozyme concentrations higher than the preferred 200 μM, the amount of 18mer product begins to decrease in the example reactions (FIG. 5).

However, the skilled practitioner can and should determine satisfactory conditions on a case-by-case basis using the examples provided herein for guidance. For example, it will be appreciated that a decrease in yield at higher ribozyme concentration could be due to the insert and starting material not binding the same ribozyme. The product yield may be increased if the insert concentration is varied in proportion with the ribozyme concentration. Time course studies do not support the possibility that higher concentrations of ribozyme may lead to TIS product breakdown. TIS reactions run with insert concentrations higher than the optimum (1 μM) also show a decrease in 18mer product formation. This could be due to the 12mer starting material not competing as well for binding the IGS in the presence of increased 9mer insert concentrations.

The preferred MgCl₂ range is about 10-14 mM MgCl₂ (FIG. 5) although higher and lower concentrations may produce acceptable results. Decreased TIS product at higher MgCl₂ concentrations in the example reactions may be due to increased strength of binding of the substrates to the ribozyme, since Mg²⁺ ions allow for tighter binding. Therefore, the MgCl₂ concentrations can be adjusted in accordance with expected relative binding depending on the IGS sequences used.

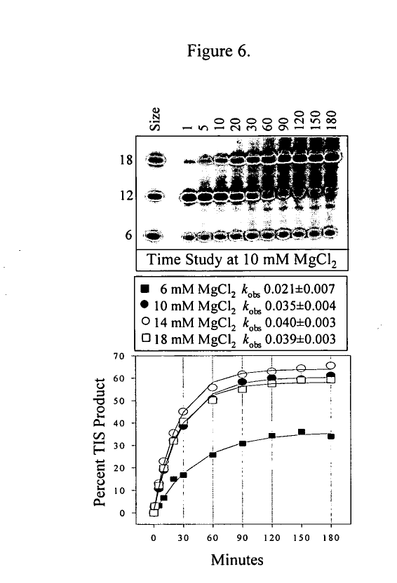

The reactions were characterized (FIG. 6) by performing time studies of rates at different MgCl₂ concentrations. The exemplary two-substrate TIS reaction is substantially completed in about two hours. Of course, the skilled practitioner can routinely determine the completion time for a TIS reaction under particular conditions. No degradation of the TIS product is seen at extended reaction times. This indicates that the TIS product is stable once it forms. The same observed rate constant is observed at 10, 14, and 18 mM MgCl₂ concentrations. The yield and observed rate constant were half the maximal values when the TIS reaction was run at 6 mM MgCl₂. Reactions run with less than preferred MgCl₂ concentration may not provide for fully folded ribozyme.

Comparing the observed rate constants for the TIS and TES reactions, the observed rate constant for the TIS reaction (0.04 min⁻¹) is 80-fold lower than for the TES reaction (3.2 min⁻¹). The TIS reaction binds two exogenous substrates (in succession) and proceeds through three catalytic steps. In comparison, the TES reaction binds one exogenous substrate and proceeds through two catalytic steps. In addition, large conformational changes are likely a part of the mechanism of the TIS reaction that are not part of the mechanism for the TES reaction.

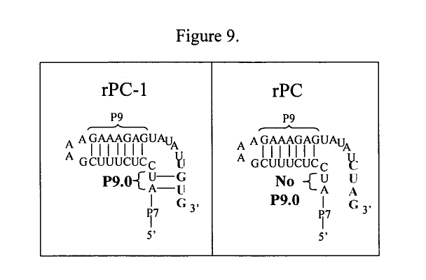

A 6-fold decrease in product yields for the TIS reaction with the rPC-1 ribozyme (compared to the rPC ribozyme with no P9.0) suggests that P9.0 formation in the ribozyme can inhibit the TIS reaction. A conformational shift between the first and second nucleophilic attacks is suggested by the mechanism to disrupt the P1i helix and form the second P1 helix. In group I introns, the P9.0 helix forms with the two nucleotides preceding ωG of the intron and can help position the ωG into the GBS (27-29). In addition, the GBS in the ribozyme binds both the exogenous G and ωG for the first and second nucleophilic attacks, respectively (30). Sullenger has shown that a Tetrahymena group I intron-derived ribozyme has different affinities for binding either the exogenous G or the ωG in the GBS between the two steps of trans-splicing (31). A similar change in affinity for binding ωG or ωGi in the GBS is expected to have a role in the TIS reaction. In the model mechanism, ωG on the ribozyme is expected to interact with the GBS in the first step of the reaction, although no P9.0 forms with this rPC ribozyme (FIG. 9). After the first step of the TIS reaction, the insert is attached to the ribozyme and the formation of a P9.0 helix at this point could help position the ωGi of the insert in the GBS for the second step of the TIS reaction (see FIG. 7C). If the ribozyme forms a P9.0 in the first reaction step, this could inhibit the second step of the TIS reaction by preventing the ωGi from interacting with the GBS.

The TIS reaction has not been previously reported. The TIS reaction provides an opposite result from the TES reaction, where a segment is removed from the middle of an RNA substrate (6). However, requirements for performing the TIS reaction are different than would be suggested by a simple reversal of the TES reaction, and the TIS reaction mechanism is not simply a reverse of the TES reaction mechanism. Another ribozyme reaction, performed with an engineered twin hairpin ribozyme, results in replacement of one RNA sequence with a longer sequence, which is essentially an “insertion” product (32). The TIS reaction differs from this twin ribozyme reaction by the type of ribozyme used and also the reaction mechanism. Moreover, the twin ribozyme reaction is reported to be much less efficient, taking 30 hours and producing 30% product compared to the exemplary results of the TIS reaction described herein.

Other ribozyme reactions have been observed in which intronic sequences can be spliced into RNA transcripts. For example, reverse-splicing occurs when a group I intron splices back into an RNA transcript (33). Group II introns can act as mobile genetic elements by splicing out of an RNA transcript, and then reverse-splicing into DNA (reviewed in (34)). These reactions differ from the TIS reaction because the inserted fragment is an exogenous substrate in TIS, not the intron itself.

Other group I intron-derived ribozyme reactions have taken advantage of ωG. A Tetrahymena intron was found to act as an enzyme by using its ωG to cleave and rejoin pentacytidylic acid, synthesizing a polycytidylic acid product (24). A recombination reaction performed by Lehman (23) and a polymerization reaction by Burke (21, 22) have an ωG in the ribozyme catalyzing a nucleophilic attack similar to the first step of the TIS reaction. In the recombination reaction, the substrates AB and CD form CB and AD (23), but do not continue polymerizing because there is no ωG on the resultant products. In the polymerization reaction, the substrate AB can form ABB, ABBB, and A(B)ₙ (22) because the 3′-G on the end of B allows it to mimic the 3′-end of the ribozyme. The TIS reaction occurs, as compared to a repeated polymerization, in part because one substrate has a 3′-G (ωGi on the insert) and one does not (starting material).

The P. carinii ribozyme recognizes two TIS substrates; initially and primarily through base pairing. The recognition elements of the ribozyme IGS can be changed to target other substrates, as has been demonstrated with the P. carinii ribozyme in the TES reaction (6). Thus, the TIS reaction can be generalized to target any desired sequence within the functional constraints of starting and targeting material. In the native state, the recognition sequence of a group I intron is complementary to a sequence at the exon splice site. Therefore, in modifying a group I intron to target a desired sequence, the IGS can be identified by locating these complementary sequences. In the two-substrate mode of the TIS, both starting material and insert substrates must bind to segments of the IGS and the insert must have a 3′ G.

For the two-substrate mode of the TIS reaction, the relative binding affinities of the target sequence and insert sequence are preferably balanced. If the P1i helix is too strong compared to the P1 helix, the starting material can be inhibited from displacing the insert from the IGS of the ribozyme. This would slow the reaction at the first step. Conversely, if the P1 helix is too strong, it might compete for binding and inhibit formation of P1i, leading to the pathway in FIG. 7E.

The TIS reaction can be utilized as biochemical tool, for example to insert a sequence, perhaps a sequence containing a modified nucleotide or a marker, into a large RNA transcript. Large RNA transcripts are typically synthesized in vitro by T7 run-off transcription, so adding site specific modifications is arduous (35). Using the TIS reaction, a small RNA insert could be synthesized (e.g. with a desired modification), targeted to an exact location, and inserted into a large transcript. The TIS reaction could also be used as an RNA repair agent. Deletion and frame shift mutations could potentially be repaired by insertion of an RNA sequence at a specific location.

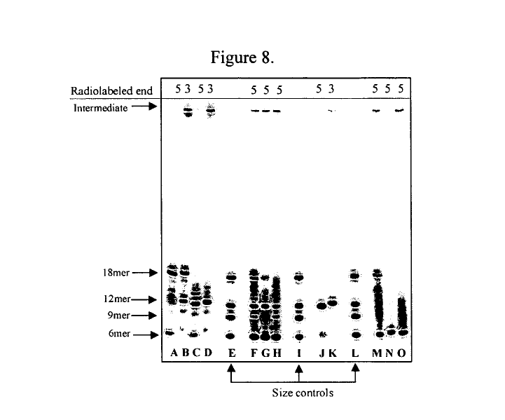

Many of the substrate sequence constraints of the two-substrate mode of the TIS reaction can easily be overcome by using a TIS ribozyme with the first intermediate already attached, as exemplified in step C of FIG. 7. The reaction then requires only a single substrate and proceeds through two reaction steps (instead of three). Results of an exemplary one-substrate reaction are shown in FIG. 8, lane F. For the one-substrate reaction, the insert need not be capable of binding to the IGS as the ribozyme is prepared with the insert attached. The insert must only comprise a 3′ G. The IGS of the ribozyme need only bind specifically to the target sequence, defining the insertion site by a wobble pair (i.e. a base pairing mismatch).

The TIS reaction in either the one-substrate mode or the two-substrate mode can be performed in vitro. Further, group I intron derived ribozymes can be for use in the reaction can be made in vitro or in vivo. For example, an isolated group I intron derived ribozyme, modified for the TIS reaction can be made from isolated DNA encoding the ribozyme by in vitro transcription using standard methods. For a one-substrate TIS reaction, ribozyme comprising an insert sequence connected to the ωG at the 3′ end of the ribozyme can be prepared by modifying a DNA plasmid encoding the ribozyme, followed by in vitro transcription using standard methods. Alternatively, the ribozyme could be prepared synthetically. A ribozyme for a one-substrate TIS reaction could be prepared by ligating an insert sequence to an isolated ribozyme using RNA ligase.

The TIS reaction can be utilized to modify RNA sequences in vivo by inserting a nucleic acid encoding the ribozyme into a cell. The nucleotide that encodes the ribozyme is preferably in an expression cassette, operably connected to sequences capable of directing expression of the ribozyme in the cell. Such an expression cassette may comprise linear DNA, contained on a plasmid, or inserted into a viral genome. The cell can be isolated cells in a cell culture or cells of an organism, including plants, animals and humans. There are a wide variety of recognized methods by which this could be accomplished, including the use of naked DNA, transfection agents such as liposomes, or any other recognized transfection techniques. The nucleotide sequence may be inserted into a viral vector, including integrating and non-integrating DNA and RNA viruses. Preferably the virus used to transfer a nucleic acid encoding the ribozyme into cells of an organism is an attenuated or replication defective virus. A host cell comprising the nucleic acid encoding the ribozyme sequence will be able to manufactures the TIS ribozyme.

In this manner, the TIS reaction can be used therapeutically to repair mRNA of the host that has a deletion mutation without relying on exogenous copies of the correct gene sequence. The endogenous gene remains under control of its native regulatory sequences. Preferably, where the TIS reaction is performed in vivo the ribozyme is transcribed from DNA that encodes the insert sequence appended to the 3′ end of the ribozyme so that only a single substrate is required for the reaction. Alternatively, an exogenous nucleic acid also provides for transcription of the insert substrate.

Group I intron-derived ribozymes obtained from various organisms can catalyze this new trans-insertion-splicing reaction. The sequence of any ribozyme can be easily manipulated such that it targets and acts upon desired substrates, including those of medical importance. Trans-insertion-splicing ribozymes are a new class of ribozymes that permit potential biochemical and therapeutic strategies not before possible.

TIS ribozymes can be introduced into and/or expressed in a host cell. Transcription of a TIS ribozyme in a host cell occurs after introduction of a ribozyme gene into the host cell. If the stable retention of the ribozyme by the host cell is not desired, the ribozyme may be provided to the host cell. Alternatively, when stable retention of the gene encoding the ribozyme is desired, such retention may be achieved by stably inserting at least one DNA copy of the ribozyme into the host's chromosome, or by providing a DNA copy of the ribozyme on a plasmid that is stably retained by the host cell. Preferably the ribozyme of the invention is inserted into the host's chromosome as part of an expression cassette, which provides transcriptional regulatory elements that control the transcription of the ribozyme in the host cell. Such elements may include, but not necessarily be limited to, a promoter element, an enhancer or UAS element, and a transcriptional terminator signal. Polyadenylation is not necessary as the ribozyme is not translated.

Expression of a ribozyme whose coding sequence has been stably inserted into a host's chromosome can be controlled by a promoter sequence that is operably linked to the ribozyme coding sequences. A promoter that directs expression of the ribozyme can be any promoter that will function in the host cell, prokaryotic promoters are preferred for use in prokaryotic cells and eukaryotic promoters in eukaryotic cells. A promoter can comprise a plurality of discrete modules that direct the transcriptional activation and/or repression of the promoter in the host cell. Such modules may be mixed and matched in a promoter so as to provide for desired expression of the ribozyme in the host. A eukaryotic promoter can be any promoter functional in eukaryotic cells, for example any having RNA polymerase I, II or III specificity. If it is desired to express the ribozyme in a wide variety of eukaryotic host cells, a promoter functional in most eukaryotic host cells should be selected, such as a rRNA or a tRNA promoter, or the promoter for a widely expressed mRNA such as the promoter for an actin gene, or a glycolytic gene. If it is desired to express the ribozyme only in a certain cell or tissue type, a cell-specific (or tissue-specific) promoter element that is functional only in that cell or tissue type should be selected. In preferred embodiments, regulatory sequences of the expressing cassette may be copies of the regulatory sequences of the gene to be affected so that transcription of the TIS ribozyme corresponds with transcription of the target RNA.

The trans-insertion-splicing reaction is chemically the same whether it is performed in vitro or in vivo. However, in vivo, the presence of the target and insertion substrates and the ribozyme will suffice to result in trans-insertion-splicing, since cofactors are already present in the host cell. However, in order to reduce the complexity of the reaction, it may be desirable to utilize a one-substrate TIS reaction wherein a TIS ribozyme is provided containing the insertion sequence appended to its 3′ ωG.

There are a vast number of pathological conditions caused by deletion mutations that could be addressed by therapeutic application of the TIS reaction. As examples, a common 3 base pair deletion can cause familial hypercholesterolemia in Ashkenazi Jews of Lithuanian descent. Meiner et al., Am J Hum Genet. 49(2):443-9, 1991. Deletion of the codons for amino acids 254-277 in the lysosomal acid lipase (LAL) mRNA can cause cholesteryl ester storage disease. Klima et al. J Clin Invest. 92(6): 2713-2718, 1993. Hereditary Hyperferritinemia-Cataract Syndrome can be caused by a 29-base pair deletion in the iron responsive element of ferritin L-subunit mRNA. Girelli et al. Blood, 90:2084-2088, 1997. In using the TIS reaction to replace a missing segment of a defective mRNA it will not be necessary to restore the precise native sequence in order to substantially restore functionality in most cases. For example, where the deletion is in the coding region, a replacement of sequence that is equivalent under the genetic code will usually be functionally equivalent. Where a deletion has caused a frame shift or missense mutation, an insert that simply restores the proper reading frame can probably restore native or near-native functionality in the majority of cases.

Alternatively, there are instances where it would be desirable to terminate expression from mRNA. For example, in some cancers a pathological mutation or translocation has eliminated a stop codon such that translation runs through from one gene to another. Translation from mRNA may be terminated by insertion of a stop codon into a target sequence in the mRNA coding sequence. Accordingly, a TIS ribozyme may be designed to insert a stop codon from an insert substrate into a target sequence in a target substrate. For a two-substrate TIS reaction, the ribozyme IGS sequence will comprise a segment that is complimentary to a stop codon contained on the insert substrate. For a one-substrate TIS reaction, the stop codon may be contained on an insert sequence that is appended to the ωG of a TIS ribozyme.

### Definitions

The following definitions are used herein.

Ribozyme: An RNA molecule that possesses catalytic activity.

Trans-splice: A form of genetic manipulation whereby a nucleic acid sequence of a first polynucleotide is colinearly linked to or inserted colinearly into the sequence of a second polynucleotide, in a manner that retains the 3′-5′ phosphodiester linkage between such polynucleotides. By “directed” trans-splicing or “substrate-specific” trans-splicing is meant a trans-splicing reaction that requires a specific RNA as a substrate for the trans-splicing reaction (that is, a specific specie of RNA in which to splice the transposed sequence). Directed trans-splicing may target more than one RNA species if the ribozyme is designed to be directed against a target sequence present in a related set of RNAs.

Trans insertion splicing (TIS): A form of trans-splicing whereby a nucleic acid sequence of a first polynucleotide is inserted colinearly into the sequence of a second polynucleotide. In its full form, TIS is a two-substrate reaction, inserting an insert sequence from an insert substrate directly into a target sequence on a target substrate. A one-substrate form of TIS is also described, wherein the ribozyme is provided with the nucleic acid sequence of a first polynucleotide appended to its 3′ end. This TIS ribozyme performs a one-substrate insertion of the insert sequence from its 3′ end into a target sequence on a target substrate.

Target substrate: A nucleic acid molecule, e.g., RNA, that is a substrate for the catalytic activity of a TIS ribozyme. In relation to the TIS reaction, the target substrate comprises a target sequence, which comprises the splice site into which an insert sequence is inserted. The target sequence consists of residues of the target substrate that are complimentary to residues in the IGS of the TIS ribozyme and a residue within the target sequence that makes a wobble pair with the IGS and that defines the insertion splice site.

Insert substrate: A nucleic acid molecule, e.g., RNA, that is a substrate for the catalytic activity of a TIS ribozyme. In relation to the TIS reaction, an insert substrate comprises an insert sequence, which is inserted in the target sequence. The insert substrate may also comprise a segment of residues that are removed in the TIS reaction. In a one-substrate TIS reaction, the ribozyme can be provided with the insert sequence appended to the ωG the 3′ end of the ribozyme.

ωG is the last G on the 3′ end of a TIS ribozyme which follows the P9.0 base pair of a group I intron. ωGi refers to a required G on the 3′ end of the insert sequence.

Internal Guide Sequence (IGS): A sequence of residues at the 5′ end of the TIS ribozyme which directs the specific binding of substrate. In a group I intron, the location of the IGS may be identified by finding a sequence of residues that is complementary to residues at the end of the corresponding exon. In a TIS ribozyme for use in the methods described herein, the IGS sequence is modified from its native sequence to specifically bind the target substrate, and in the two-substrate TIS reaction to bind the insert.

Wobble pair: A pair of nucleotides in otherwise complementary segments of nucleic acid molecules that have a nonstandard base pairing. In the context of translation of the genetic code, wobble pairing generally occurs in the third position of a codon. However, as used herein, wobble pairs are not limited to the third position of the codon, rather, wobble pair refers to a base pair mismatch in the complementary segments of substrate that bind to the IGS of a TIS ribozyme. These wobble pairs define the splice site.

Expression Cassette: A genetic sequence that provides sequences necessary for the expression of a ribozyme of the invention.

Stably: By “stably” inserting a sequence into a genome is intended insertion in a manner that results in inheritance of such sequence in copies of such genome.

Operable linkage: An “operable linkage” is a linkage in which a sequence is connected to another sequence (or sequences) in such a way as to be capable of altering the functioning of the sequence (or sequences). For example, by operably linking a ribozyme encoding sequence to a promoter, expression of the ribozyme encoding sequence is placed under the influence or control of that promoter. Two nucleic acid sequences, such as a ribozyme encoding sequence and a promoter region sequence at the 5′ end of the encoding sequence, are said to be operably linked if induction of promoter function results in the transcription of the ribozyme encoding sequence and if the nature of the linkage between the two sequences does not (1) result in the introduction of a frame-shift mutation, (2) interfere with the ability of the expression regulatory sequences to direct the expression of the ribozyme. Thus, a promoter region would be operably linked to a nucleic acid sequence if the promoter were capable of effecting the synthesis of that nucleic acid sequence.

Native target sequence/Non-native target sequence: Native target sequence of a ribozyme is that polynucleotide sequence which is recognized, bound and reacted by wild-type (native) ribozymes. Non-native target sequence is sequence within a substrate that is not bound by wild type ribozyme and consequently native ribozyme does not react with non-native target sequence. A TIS ribozyme for use in the methods described herein will have a different target sequence than

An isolated nucleic acid, such as an isolated ribozyme, is a nucleic acid which is not connected to the sequences to which it is connected in its native state or is an artificially constructed nucleic acid. A ribozyme derived from a group I intron refers to a group I intron which has been isolated or is made by transcription from an isolated nucleic acid and which has been modified to have a non-native targeting sequence.

While the invention has been described in detail with reference to preferred embodiments thereof, it will be apparent to one skilled in the art that various changes can be made, and equivalents employed, without departing from the scope of the invention. The following examples demonstrate the operation of a TIS reaction and the characterization of reaction conditions for one exemplary embodiment, but should not be construed as limiting in any manner.

### EXAMPLES

### Materials and Methods used in Example Experiments

Synthesis and Preparation of Oligonucleotide. RNA oligonucleotides were purchased from Dharmacon Research, Inc. (Lafayette, Colo.) and deprotected according to the manufacture's instructions. Oligonucleotide concentrations were calculated based on UV-absorption measurements using a Beckman DU 650 UV-Spectrophotometer (Beckman Coulter, Inc., Fullerton, Calif.). Designated oligonucleotides were 5′-end radiolabeled and purified by gel electrophoresis as described previously (18).

Labeled oligonucleotides were 3′-end radiolabeled by ligating 5′-end radiolabeled Cp to the 3′-end of the oligonucleotide. The Cp was 5′-end radiolabeled in 10 μL consisting of 5 μL of 250 μCi ³²P-δATP (Amersham-Pharmacia, Piscataway, N.J.), 2 μL of 50 mM cytidine 5′ monophosphate (CMP) (Sigma, St. Louis, Mo.), 1 μL of 10 U/μL T4 poly-nucleotide kinase (New England Biolabs, Beverly, Mass.), and 1 μL of 10× poly-nucleotide kinase buffer (supplied by New England Biolabs). The reaction was run for 90 min at 37° C., and then the kinase was deactivated by incubation at 65° C. for 15 min. 5′-end radiolabeled pCp (5′-*pCp) was ligated to the 3′-end of the oligonucleotide in a 10 μL reaction mixture consisting of 1 μL of DMSO, 2 μL of 10 μM RNA, 2 μL of 20 U/μL T4 RNA ligase (New England Biolabs), 4 μL of 5′-*pCp (approximately 20 μM), and 1 μL of 10× T4 RNA ligase buffer (New England Biolabs). The reactions were incubated for 16 h at 4° C. The 3′-radiolabeled oligonucleotides were purified by gel electrophoresis as described for the 5′-end radiolabeled oligonucleotides. The substrate names and sequences are shown in Table 1.

TABLE 1
Ribozyme, starting material, and insert sequences. rPC-3, rPC-3ωA, rPC-
4, and 12 mer disclosed as SEQ ID NOS 8-10 and 3, respectively.

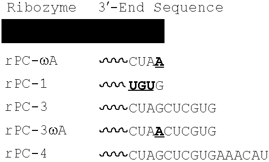

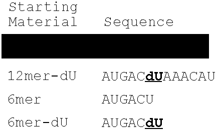

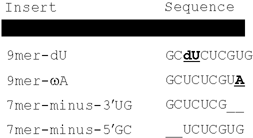

The sequences highlighted by gray boxes indicate
the substrates and ribozyme used in the standard
TIS reaction. The bold and underlined nucleotides
indicate the positions that differ from the standard
reaction components. Note that rPC-3, rPC-3ωA,
and rPC-4 are the intermediate ribozymes with part
of the starting material and insert attached to the

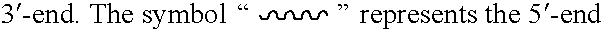

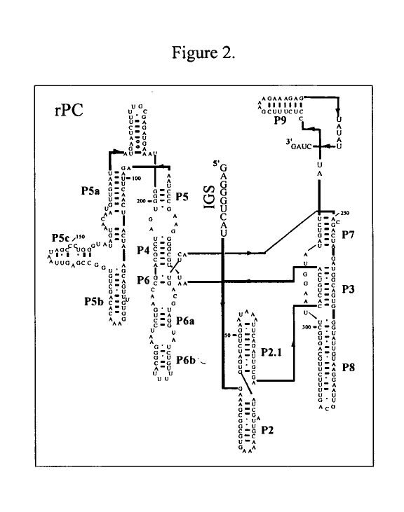

of the ribozyme that is the same for all the
ribozymes tested. The complete ribozyme
sequence is shown in Figure 2.

Ribozyme Preparation. The P. carinii (PC) ribozyme plasmid (proposed secondary structure of the rPC ribozyme shown in FIG. 2) was linearized in a 50 μL reaction mixture consisting of 8 μg of plasmid, 50 U of XbaI (Invitrogen, Grand Island, N.Y.), and 1× React 2 buffer at 37° C. for 2 h. Linearization was confirmed by visualization on a 1% agarose gel. The linearized DNA was purified using a QIAquick PCR Purification Kit (Qiagen Inc., Valencia, Calif.) and eluted in water. Five other ribozymes with varying 3′-ends were made from the PCR products derived from the PC plasmid (Table 1). The upstream PCR primer for all five ribozymes was ^{5′}CTCTAATACGACTCACTATAGAGGG³ (SEQ ID NO: 11). The following sequences are the downstream primers for each ribozyme (the variable region is underlined): ^{5′}CACAATATACTCTTTCTTTCGAAAGAGG^{3′} (SEQ ID NO: 12) for rPC-1, ^{5′}TTAGATATACTCTTTCTTTCGAAAGAGG^{3′} (SEQ ID NO: 13) for rPC-ωA, ^{5′}CACGAGCTAGATATACTCTTTCTTTCGAAAGAGG^{3′} (SEQ ID NO: 14) for rPC-3, ^{5′}CACGAGTTAGATATACTCTTTCTTTCGAAAGAGG^{3′} (SEQ ID NO: 15) for rPC-3-ωA, and ^{5′}ATGTTTCACGAGCTAGATATACTCTTTCTTTCGAAAGAGG^{3′} (SEQ ID NO: 16) for rPC-4. The PCR products were gel purified using a Qiagen gel extraction kit (Qiagen Inc.). Run-off transcription was performed for 2 h in 100 μL reactions consisting of 1-2 μg of linear DNA, 50 U of T7 RNA polymerase (New England Biolabs), 40 mM Tris-HCl (pH 7.5), 10 mM MgCl₂, 5 mM DTT, 5 mM spermidine, 1 mM rNTP mix, and 62.5 μg/mL BSA. The ribozymes were purified using a Qiagen RNeasy purification kit (Qiagen Inc.).

The Trans Insertion-Splicing Reaction. The extent of the TIS reaction was characterized over a range conditions under which the reaction can be performed, including rPC concentration (10 to 1000 nM), 9mer insert concentration (10 to 3000 nM), MgCl₂ concentration (2 to 15 mM), time (1 to 180 min), and temperature (37° to 50° C.) for the reaction with 5′-end radiolabeled 12mer starting material AUGACUAAACAU (SEQ ID NO: 3). Preferred reaction conditions were found to include of 2 h of reaction time at 44° C. with 200 nM rPC, 1 μM 9mer insert, and 10 mM MgCl₂. All reactions were run using approximately 1 nM 5′-end radiolabeled 12mer starting material in a buffer consisting of 50 mM Hepes (25 nM Na⁺) and 135 mM KCl at pH 7.5.

In the reactions, first 3 μL of ribozyme in appropriate buffer was pre-annealed at 60° C. for 5 min. The ribozyme was then slowly cooled to 44° C. Reactions were initiated by adding 2 μL of pre-combined substrates (radiolabeled starting material and cold insert). Optimum TIS reactions were run with 5′-end radiolabeled 12mer starting material, but 3′-end radiolabeled starting material and 5′ and 3′-end radiolabeled insert were also used in reactions to elucidate the mechanism. Reactions were terminated after 2 h by adding 5 μL stop buffer (10 M urea, 3 mM EDTA, 0.1× TBE). The reactions were denatured for 1 min at 90° C. and then separated on a 12% polyacrylamide/8M urea gel. The gel was transferred to chromatography paper and dried under vacuum. The bands were visualized and quantified on a Molecular Dynamics Storm 860 Phosphorimager. The observed rate constant, k_{obs}, for the TIS reaction was obtained from the plot of percent TIS product formed over time (18).

Product Isolation and Identification. The TIS product was gel purified and sequenced by partial nuclease digestion along with the synthetic version of the expected product. The TIS reaction was scaled up 50-fold (250 μL) and run as described above using the optimized conditions, except the reactions were run in five 50 μL volumes, concentrated to 30 μL after 2 h, and terminated with 10 μL stop buffer. The product band was ultimately cut out of the gel and eluted from the gel matrix for 40 min by crushing with a stir bar in 400 μL of elution buffer (0.3 M sodium acetate, 5 mM EDTA, 10 mM Tris-HCl pH 7.5, and 0.1% SDS). The eluent was decanted and a second round of elution was performed. The product was ethanol precipitated overnight and concentrated to 30 μL. The radiolabeled TIS product and synthetic 18-mer were enzymatically sequenced using RNA nucleases T1, U2, CL-3 and, B. cereus (Research Unlimited; Wellington, New Zealand) essentially as described (6). B. cereus reactions used 0.33 units B. cereus in 33 mM sodium citrate (pH 5.0) and 1.7 mM EDTA.

### Experimental Results

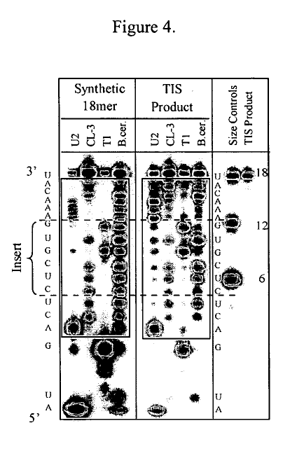

Group I Introns Catalyze the TIS Reaction. In initial experiments, reactions with 5′-end radiolabeled 12mer starting material, non-labeled 9mer insert, and the ribozyme rPC (secondary structure shown in FIG. 2) showed a potential insertion (FIG. 3, 18mer in lane A), but it was not the size that would have been expected from a simple reversal of the TES reaction (FIG. 1A). An experiment was conducted to test whether this product also contained the 3′-terminal region of the 12mer starting material by running the reaction independently with 3′-end radiolabeled 12mer starting material (FIG. 3, 18mer in lane B). A higher sized product was produced, because the 3′-end radiolabeled starting material is one nucleotide larger than the 5′-end radiolabeled starting material due to the method of labeling. This product contains both the 5′ and 3′-ends of the starting material, and demonstrates that an insertion reaction occurred. For confirmation, the product band was excised from the polyacrylamide gel and its sequence was confirmed by enzymatic sequencing (FIG. 4). The 18mer product AUGACUCUCGUGAAACAU (SEQ ID NO: 2) is the result of insertion of the underlined 6mer region from the 9mer insert, GCUCUCGUG, into the middle of the 12mer starting material, AUGACUAAACAU (SEQ ID NO: 3). Therefore, although the exact product was unexpected, this is the first experimental result proving that group I intron-derived ribozymes can catalyze a TIS reaction.

The exemplary reactions were characterized in terms of the yield (FIG. 5) and the observed rate constant (FIG. 6) under various conditions. Preferred reaction conditions for two-substrate TIS are 200 nM ribozyme (rPC), 1 μM 9mer insert, and 10 mM MgCl₂ using 1 nM radiolabeled 12mer starting material (see FIG. 5). Under such conditions, this exemplary TIS reaction can produce 62.9±1.3% 18mer TIS product in about two hours. Studies of the course of the exemplary reaction over time were performed at four different MgCl₂ concentrations and the observed rate constants, k_{obs}, were determined (FIG. 6). The k_{obs} value for the exemplary TIS reaction is 0.04 min⁻¹, and is the same at 10, 14, and 18 mM MgCl₂. The k_{obs} value when using 6 mM MgCl₂, however, was 0.02 min⁻¹, which is half the k_{obs} for the optimum condition and results also in half the yield. Apparently MgCl₂ concentrations of 6 mM or less can inhibit the ability of the ribozyme to fold, as well as to catalyze the reaction.

A mechanism for the TIS reaction has been deduced by analyzing the effects of modifying functional groups in the substrates and ribozyme, and by initiating the reaction using intermediates as starting material. Of note is that a large intermediate product, indicative of the substrate attached to the ribozyme, was observed only when substrates were 3′-end radiolabeled (not when they were 5′-end radiolabeled). This indicates that the ωG on the ribozyme is involved in catalysis and that intermediates are attached to the ribozyme during the reaction. A mechanism for TIS that is supported by results of experiments performed as described below is shown in FIG. 7.

Step 1: The ωG on the Ribozyme Performs a Nucleophilic Attack on the Insert. The sequencing results show that the 9mer insert is losing its 5′-GCU (GCUCUCGUG) during the TIS reaction. The loss of this GCU 3mer from the insert suggests that the insert might first be forming a P1 helix (called P1i) with the IGS of the ribozyme (FIG. 7B). This interaction would place a G-U wobble pair at the 5′-splice site where the ωG on the ribozyme could attack the insert.

To determine if the ribozyme's ωG is involved in the mechanism proposed in FIG. 7, the ωG in ribozyme rPC was replaced with an ωA (rPC-ωA). Although an ωA has been shown to allow the second step of splicing in a group I intron from Anabaena (19), an adenosine was used to test the TIS reaction because the GBS in the P. carinii ribozyme has does not functionally interact with adenosine for the second step of the TES reaction (17). If the ωG in the ribozyme participates in the first step, its alteration would affect or prevent the first reaction step, as well as TIS product formation. In this reaction, using 5′ or 3′-end radiolabeled 12mer starting material, no appreciable 18mer TIS product formed (FIG. 3, lanes C and D). Note that in the 5′-end radiolabeled reaction (FIG. 3, lane C), production of 6mer is occurring through ribozyme-mediated hydrolysis at the 5′-splice site, as seen in the TES reaction with the same ribozyme (6). Also, in the 3′-end radiolabeled reaction (FIG. 3, lane D), much less of the large intermediate band forms compared to the reaction using rPC (compare to FIG. 3, lane B), as expected if the ribozyme cannot perform the first step of the TIS reaction.

To determine if the 9mer insert is forming a P1i helix (as depicted in FIG. 7B), the 9mer insert was shortened on either the 5′ or 3′-end. The 7mer-minus-3′UG insert (GCUCUCG_{——}) is shortened by two nucleotides on its 3′-end. This insert can still form a P1i helix, but should give a shortened TIS product. Reactions run with 5′-end radiolabled 12mer starting material, rPC ribozyme, and 7mer-minus-3′UG insert (FIG. 3, lane G) show production of a shorter TIS product.

An insert was shortened by two nucleotides on its 5′end, 7mer-minus-5′GC insert (_{——}UCUCGUG), which should disrupt P1i formation. If relatively stable P1i formation is required, then this insert will not form TIS product. Reactions run with 5′-end radiolabled 12mer starting material, rPC ribozyme, and 7mer-minus-5′GC insert (FIG. 3, lane F) show no TIS production, which indicates that the insert is forming a P1i helix in the TIS reaction.

Since the 9mer insert loses its 5′GCU and appears to form a P1i helix, an insert with a deoxy-U at the critical position forming the 5′-splice site (9mer-dU, GCdUCUCGUG) was tested to determine if this change inhibits 18mer production. If catalysis is occurring at this position in the 9mer, the substitution of a deoxy-U should either prevent or greatly inhibit TIS. The reaction run with 5′-end radiolabled 12mer starting material, rPC ribozyme, and the 9mer-dU insert gives greatly inhibited TIS production (FIG. 3, lane H).

To show directly that the ribozyme is attacking the 9mer insert, reactions were run with 5′-end radiolabeled insert instead of radiolabeled starting material. These reactions were run with much less insert compared to the standard reaction, and were accordingly not under optimum TIS conditions. Nevertheless, when the reaction is run with 5′-end radiolabeled 9mer (GCUCUCGUG), all of the 9mer is cleaved to a smaller product (FIG. 3, lane J), which appears to be the 3mer side product GCU. Running the same reaction using the ribozyme rPC-ωA and 5′-end radiolabeled 9mer gives a marked reduction in 3mer formation (FIG. 3, lane K). The 3mer produced in this reaction is believed to be mostly attributable to ribozyme-mediated cleavage since the ωA in the ribozyme does not attack at the splice site.

When the reaction was run with 3′-end radiolabeled 9mer insert (also not run with optimum TIS insert concentrations), a large intermediate band forms (FIG. 3, lane M). This band is intermediate formed by the insert attaching to the 3′-end of the ribozyme (shown in FIG. 7C). The formation of this large intermediate band was greatly inhibited when 3′-end radiolabeled 9mer is run with rPC-ωA (FIG. 3, lane O). Likewise, this inhibition also occurs when running the reaction with 3′-end radiolabeled 9mer-dU (FIG. 3, lane N). These results indicate that the 9mer loses the 3mer (5′GCU) from its 5′-end and the 3′-portion of the insert becomes ligated to the 3′-end of the ribozyme in the process (FIG. 7).

Taken together, these results indicate that the 9mer insert binds the IGS of the ribozyme, forming a P1i helix. The P1i helix positions the G-U wobble pair at the 5′-splice site, and the ωG on the ribozyme performs the first nucleophilic attack at this position.

Step 2: ωGi on the Insert Performs a Nucleophilic Attack on the Starting Material. Once the insert fragment is attached to the 3′-end of the ribozyme, the 12mer starting material binds the ribozyme and forms a P1 helix. The 3′-G from the insert (called ωGi) now acts as the nucleophile and can attack the G-U wobble pair at the new 5′-splice site in the starting material (FIG. 7C).

The importance of the ωGi on the 9mer insert was tested by changing ωGi to an ωAi (GCUCUCGUA, called 9mer-ωAi). If ωGi is interacting with the GBS and acting similar to the ωG on the ribozyme, then changing this base to an ωA should prevent the TIS reaction. Running the TIS reaction with 5′-end radiolabled 12mer starting material, rPC ribozyme, and the 9mer-ωA insert gives no 18mer TIS product (FIG. 8, lane C). For comparison, the standard TIS reaction is shown in FIG. 8, with the 5′-end radiolabeled reaction in lane A and the 3′-end radiolabeled reaction in lane B. The reaction run with 3′-end radiolabeled 12mer starting material, rPC ribozyme, and 9mer-ωA insert also shows no TIS product (FIG. 8, lane D). Evidently, ωGi on the insert is necessary to the TIS reaction. Since the rPC ribozyme has an ωG, the 6mer (FIG. 8, lane C) and the large intermediate (FIG. 8, lane D) are likely due to the competing reaction depicted in FIG. 7E.

To demonstrate a one-substrate variation of the method, the TIS reaction was initiated at the second step of the three-step reaction by reacting 12mer starting material with an intermediate ribozyme (rPC-3) that has the insert fragment already attached to its 3′-end (FIG. 7C). This TIS reaction was run exactly like the standard TIS reaction, except no 9mer insert was added. As seen in FIG. 8, lane F, the reaction of the rPC-3 ribozyme and 5′-end radiolabeled 12mer starting material produces 18mer TIS product. Since no insert is added to the reaction, the inserted sequence must be coming from the 3′-end of the ribozyme.

In our proposed mechanism, the second step of the TIS reaction occurs via a nucleophilic attack on the 5′-splice site of the 12mer starting material. This was tested by changing the ribo-U at the 5′-splice site to a deoxy-U (12mer-dU, AUGACdUAAACAU (SEQ ID NO: 3)). In the reaction with 5′-end radiolabeled 12mer-dU starting material, the ribozyme intermediate rPC-3, and no added 9mer insert, no TIS product forms (FIG. 8, lane J). Likewise, the same reaction run with 3′-end radiolabeled 12mer-dU shows no TIS product (FIG. 8, lane K). Clearly, changing the ribo-U to a deoxy-U at this position in the 12mer starting material (AUGACdUAAACAU) (SEQ ID NO: 3) suggests that this position forms the second 5′-splice site.

These results indicate that in the second step of the TIS reaction, the 12mer starting material forms a P1 helix with the ribozyme, and the ωGi on the insert acts as a nucleophile in the 5′-cleavage reaction.

Step 3: The 3′-U on the 5′-half of the Starting Material Attacks the ωG of the Ribozyme, Forming TIS Product. After the second step of the TIS reaction, the 3′-half of the starting material, as well as the insert region, is attached to the 3′-end of the ribozyme (FIG. 7D). The 3′-U on the 5′-half of the starting material can then mimic the second step of self-splicing and attack at ωG (and not at ωGi) of the ribozyme. To initiate the reaction at the third reaction step, we synthesized the ribozyme intermediate rPC-4, which has the insert and 3′-half of the starting material on the 3′-end of the ribozyme (FIG. 7D), and ran the reaction using 5′-end radiolabeled 6mer (AUGACU, the 5′-half of the 12mer starting material). This reaction produces the expected 18mer TIS product (FIG. 8, lane M). Since the 18mer product forms with only 6mer added, the product sequence must be coming from the ribozyme, as proposed (FIG. 7D). The control reaction of 5′-end radiolabeled 6mer and rPC (the standard ribozyme) (FIG. 8, lane O), shows no 18mer product. In addition, running the reaction with rPC-4 and the 6mer with its 3′-ribo-U changed to a deoxy-U (AUGACdU called 6mer-dU) greatly reduces TIS product (FIG. 8, lane N), indicating that the 3′-U on the 5′-half of the starting material is the nucleophile for the third reaction step.

Finally, the third step of the reaction can be inhibited by changing the ωG in the rPC-3 ribozyme to an ωA (rPC-3ωA in Table 1). The reaction utilizing 12mer starting material and rPC-3ωA initiates at the second step of TIS (FIG. 7C), but the ωA in the ribozyme should inhibit the TIS reaction at the third step (FIG. 7D). The results show less 18mer product forms (FIG. 8, lane G), as expected, compared to the reaction with rPC-3 (FIG. 8, lane F). This result suggests that the ωG in the ribozyme is not only required for the first step, but is important for the third step of the TIS reaction as well.

From these results, it appears that the 3′-U on the 5′-half of the starting material attacks at the ωG in the ribozyme to form the TIS product.

P9.0 Formation Inhibits the TIS Reaction. The TIS reaction was also run with the native P. carinii intron sequence (called rPC-1). In reactions with rPC-1, 18mer product yields were 6-fold lower than with rPC (11.0±0.8% compared to 62.9±1.3%). The only difference between the rPC-1 and rPC is three nucleotides at the 3′-end of the ribozymes (FIG. 9). This difference allows only rPC-1 to form a P9.0 helix. Apparently P9.0 formation inhibits the TIS reaction, perhaps because it inhibits the proposed conformational shift that occurs between the first and second steps, essentially halting the reaction at the second step.

### REFERENCES

1. Kruger, K., Grabowski, P. J., Zaug, A. J., Sands, J., Gottschling, D. E., and Cech, T. R. (1982) Self-splicing RNA: autoexcision and autocyclization of the ribosomal RNA intervening sequence of Tetrahymena, Cell 31, 147-57.

2. Guerrier-Takada, C., Gardiner, K., Marsh, T., Pace, N., and Altman, S. (1983) The RNA moiety of ribonuclease P is the catalytic subunit of the enzyme, Cell 35, 849-57.

3. Doudna, J. A., and Cech, T. R. (2002) The chemical repertoire of natural ribozymes, Nature 418, 222-8.

4. Breaker, R. R. (2004) Natural and engineered nucleic acids as tools to explore biology, Nature 432, 838-45.

5. Sullenger, B. A., and Cech, T. R. (1994) Ribozyme-mediated repair of defective mRNA by targeted, trans-splicing, Nature 371, 619-22.

6. Bell, M. A., Johnson, A. K., and Testa, S. M. (2002) Ribozyme-catalyzed excision of targeted sequences from within RNAs, Biochemistry 41, 15327-33.

7. Mansfield, S. G., Clark, R. H., Puttaraju, M., Kole, J., Cohn, J. A., Mitchell, L. G., and Garcia-Blanco, M. A. (2003) 5′ exon replacement and repair by spliceosome-mediated RNA trans-splicing, RNA 9, 1290-7.

8. Mansfield, S. G., Kole, J., Puttaraju, M., Yang, C. C., Garcia-Blanco, M. A., Cohn, J. A., and Mitchell, L. G. (2000) Repair of CFTR mRNA by spliceosome-mediated RNA trans-splicing, Gene Ther 7, 1885-95.

9. Sullenger, B. A., and Gilboa, E. (2002) Emerging clinical applications of RNA, Nature 418, 252-8.

10. Puerta-Fernandez, E., Romero-Lopez, C., Barroso-delJesus, A., and Berzal-Herranz, A. (2003) Ribozymes: recent advances in the development of RNA tools, FEMS Microbiol Rev 27, 75-97.

11. Steele, D., Kertsburg, A., and Soukup, G. A. (2003) Engineered catalytic RNA and DNA: new biochemical tools for drug discovery and design, Am J Pharmacogenomics 3, 131-44.

12. Fire, A., Xu, S., Montgomery, M. K., Kostas, S. A., Driver, S. E., and Mello, C. C. (1998) Potent and specific genetic interference by double-stranded RNA in Caenorhabditis elegans, Nature 391, 806-11.

13. Mello, C. C., and Conte, D., Jr. (2004) Revealing the world of RNA interference, Nature 431, 338-42.

14. Winkler, W. C., Nahvi, A., Roth, A., Collins, J. A., and Breaker, R. R. (2004) Control of gene expression by a natural metabolite-responsive ribozyme, Nature 428, 281-6.

15. Batey, R. T., Gilbert, S. D., and Montange, R. K. (2004) Structure of a natural guanine-responsive riboswitch complexed with the metabolite hypoxanthine, Nature 432, 411-5.

16. Bell, M. A., Sinha, J., Johnson, A. K., and Testa, S. M. (2004) Enhancing the second step of the trans excision-splicing reaction of a group I ribozyme by exploiting P9.0 and P10 for intermolecular recognition, Biochemistry 43, 4323-31.

17. Baum, D. A., Sinha, J., and Testa, S. M. (2005) Molecular Recognition in a Trans Excision-Splicing Ribozyme: Non-Watson-Crick Base Pairs at the 5′ Splice Site and omegaG at the 3′ Splice Site Can Play a Role in Determining the Binding Register of Reaction Substrates, Biochemistry 44, 1067-77.

18. Testa, S. M., Haidaris, C. G., Gigliotti, F., and Turner, D. H. (1997) A Pneumocystis carinii group I intron ribozyme that does not require 2′ OH groups on its 5′ exon mimic for binding to the catalytic core, Biochemistry 36, 15303-14.

19. Golden, B. L., and Cech, T. R. (1996) Conformational switches involved in orchestrating the successive steps of group I RNA splicing, Biochemistry 35, 3754-63.

20. Cech, T. R. (1990) Self-splicing of group I introns, Annu Rev Biochem 59, 543-68.

21. Chowrira, B. M., Berzal-Herranz, A., and Burke, J. M. (1995) Novel system for analysis of group I 3′ splice site reactions based on functional trans-interaction of the P1/P10 reaction helix with the ribozyme's catalytic core, Nucleic Acids Res 23, 849-55.

22. Chowrira, B. M., Berzal-Herranz, A., and Burke, J. M. (1993) Novel RNA polymerization reaction catalyzed by a group I ribozyme, Embo J 12, 3599-605.

23. Riley, C. A., and Lehman, N. (2003) Generalized RNA-directed recombination of RNA, Chem Biol 10, 1233-43.

24. Zaug, A. J., and Cech, T. R. (1986) The intervening sequence RNA of Tetrahymena is an enzyme, Science 231, 470-5.

25. Milligan, J. F., Groebe, D. R., Witherell, G. W., and Uhlenbeck, O. C. (1987) Oligoribonucleotide synthesis using T7 RNA polymerase and synthetic DNA templates, Nucleic Acids Res 15, 8783-98.

26. Turnbull-Ross, A. D., Else, A. J., and Eperon, I. C. (1988) The dependence of splicing efficiency on the length of 3′ exon, Nucleic Acids Res 16, 395-411.

27. Burke, J. M., Esherick, J. S., Burfeind, W. R., and King, J. L. (1990) A 3′ splice site-binding sequence in the catalytic core of a group I intron, Nature 344, 80-2.

28. Watanabe, S., Kawai, G., Muto, Y., Watanabe, K., Inoue, T., and Yokoyama, S. (1996) An RNA fragment consisting of the P7 and P9.0 stems and the 3′-terminal guanosine of the Tetrahymena group I intron, Nucleic Acids Res 24, 1337-44.

29. Michel, F., Netter, P., Xu, M. Q., and Shub, D. A. (1990) Mechanism of 3′ splice site selection by the catalytic core of the sun Y intron of bacteriophage T4: the role of a novel base-pairing interaction in group I introns, Genes Dev 4, 777-88.

30. Been, M. D., and Perrotta, A. T. (1991) Group I intron self-splicing with adenosine: evidence for a single nucleoside-binding site, Science 252, 434-7.

31. Zarrinkar, P. P., and Sullenger, B. A. (1998) Probing the interplay between the two steps of group I intron splicing: competition of exogenous guanosine with omega G, Biochemistry 37, 18056-63.

32. Welz, R., Bossmann, K., Klug, C., Schmidt, C., Fritz, H. J., and Muller, S. (2003) Site-directed alteration of RNA sequence mediated by an engineered twin ribozyme, Angew Chem Int Ed Engl 42, 2424-7.

33. Woodson, S. A., and Cech, T. R. (1989) Reverse self-splicing of the tetrahymena group I intron: implication for the directionality of splicing and for intron transposition, Cell 57, 335-45.

34. Lambowitz, A. M., and Zimmerly, S. (2004) Mobile Group II Introns, Annu Rev Genet.

35. Moore, M. J., and Sharp, P. A. (1992) Site-specific modification of pre-mRNA: the 2′-hydroxyl groups at the splice sites, Science 256, 992-7.

36. Baum, D. A., and Testa, S. M. (2005) In Vivo Excision of a Single Targeted Nucleotide from an mRNA by a Trans Excision-Splicing Ribozyme, RNA “Maunscript in Press”.

What is claimed is:

1. An isolated ribozyme comprising an RNA sequence derived from a group I intron, wherein the ribozyme has a non-native IGS sequence and a 3′ ωG, and optionally wherein an insert sequence having a 3′ ωGi is appended to the 3′ ωG of the ribozyme.

2. The ribozyme of claim 1 wherein an insert sequence having a 3′ ωGi is appended to the 3′ ωG of the ribozyme.

3. A composition comprising the ribozyme of claim 1 and an RNA target substrate comprising a sequence that is complementary to a segment of the non-native IGS sequence of the ribozyme.

4. A composition of claim 3, further comprising an insert substrate that comprises an insert sequence that is complementary to a segment of the non-native IGS sequence of the ribozyme and has a 3′ G.

5. A composition comprising the ribozyme of claim 2 and an RNA target substrate comprising a sequence that is complementary to a segment of the non-native IGS sequence of the ribozyme.

6. The ribozyme of claim 1 wherein the non-native IGS sequence comprises a segment complimentary to a stop codon.

7. The ribozyme of claim 2 wherein the insert sequence comprises a stop codon.

8. The ribozyme of claim 1 wherein the ribozyme comprises a sequence of rP-8/4.

9. The ribozyme of claim 1 wherein the ribozyme is a modified P. carinii ribozyme.

10. A method of inserting an RNA insert sequence into an RNA target substrate comprising contacting the target substrate with a ribozyme of claim 1, wherein the non-native IGS sequence of the ribozyme comprises a segment that is complimentary to a residues of a target sequence of the target substrate on both sides of an insertion site except for a mismatched pair base pairing at the insertion site.

11. The method of claim 11 further comprising contacting the ribozyme with an insert substrate wherein the non-native IGS sequence of the ribozyme comprises a segment that is complimentary to a segment of an insert sequence of the insert substrate.

12. The method of claim 10 wherein the ribozyme is derived from rP-8/4x.

13. The method of claim 10, further comprising introducing a nucleic acid comprising an expression cassette which includes a sequence encoding the ribozyme into a cell, wherein the target substrate is an RNA molecule produced in the cell.

14. A method of treating a disease associated with a mutation of a gene that results in production of a non-native mRNA that is missing a segment normally found in a native mRNA produced from the gene, the method comprising
administering, to a patient possessing the mutation, a ribozyme of claim 1 wherein the IGS sequence contains a sequence complementary to residues on both sides of the site of the missing segment of the non-native mRNA and a base pair mismatch at the site of the missing segment of the non-native mRNA, and wherein the ribozyme comprises an insert sequence appended to the 3′ ωG that can restore substantial functionality to the mRNA.

15. The method of claim 14, wherein the insert sequence comprises the native sequence of the segment missing from the non-native mRNA or a segment or its genetic code equivalent.

16. The method of claim 14 wherein the insert sequence restores the reading frame of the mRNA

17. The method of claim 14 wherein the insert sequence comprises a stop codon.

18. A DNA expression cassette comprising a promoter operably-linked to an isolated nucleotide sequence encoding a ribozyme of claim 1.
